(19) United States
(12) Patent Application Publication
(10) Pub. No.: US 2006/0281813 A1
(43) Pub. Date: Dec. 14, 2006

(54) Methods of treating hyperproliferative cell disorders

(76) Inventor: Charles Rubin, North Bergen, NJ (US)

Correspondence Address: Charles C Achkar, 7855 Boulevard East #221, North Bergen, NJ, 07047, US

(21) Appl. No.: 10/558,350

(22) PCT Filed: Jun. 7, 2004
(86) PCT No.: PCT/US04/17831

§ 371 Date: Nov. 23, 2005

Related U.S. Application Data
(60) Provisional application No. 60/476,412, filed on Jun. 5, 2003.

(51) Int. Cl. A61K 31/203; A61K 31/12; A61K 31/11; A61K 31/07

(52) U.S. Cl. 514/559; 514/725; 514/690; 514/703

(57) ABSTRACT

Disclosed are methods of treating subjects suffering from hyperproliferative cell disorders which consist of administering to those subjects a combination treatment of a specific retinoid and optionally a growth factor receptor inhibitor.

### FIELD OF THE INVENTION

The present invention relates to compositions comprising certain retinoids and optionally growth factor antagonists agents useful in inducing apoptosis and differentiation as well as inhibiting undesirable proliferation of cancer. The present invention also relates to methods of using the above compositions in the treatment of diseases and conditions characterized by abnormal cell differentiation and/or cell proliferation such as cancer.

### DESCRIPTION OF THE RELATED ART

Growth Factor Pathway Inhibitors

In advanced stages of disease, many malignancies are treated with cytotoxic chemotherapy. Cytotoxic agents (e.g. antimetabolites, alkylating agents) damage DNA directly, leading to cell death. However, many such agents have considerable toxicity, frequently leading to alopecia, myelosuppression, and nausea and vomiting. In addition, clinically meaningful responses to cytotoxic agents with symptom improvement, are often disappointingly low. Increase understanding of cancer biology has led to the search for new classes of anticancer agents with greater selectivity to attain higher therapeutic ratios of efficacy over toxicity. A greater understanding of cancer cell receptors and their mitogenic signaling pathways has given rise to considerable interest in the development of signal transduction inhibitors.

The epidermal growth factor receptor (EGFR) is a member of the type 1 tyrosine kinase family of growth factor receptors, which play critical roles in cellular growth, differentiation, and survival. Activation of these receptors typically occurs via specific ligand binding, resulting in hetero- or homdimerization between receptor family members, with subsequent autophosphorylation of the tyrosine kinase domain. This activation triggers a cascade of intracellular signaling pathways involved in both cellular proliferation (the ras/raf/MAP kinase pathway) and survival (the P13 kinase/Akt pathway). Members of this family, including EGFR and Her2, have been directly implicated in cellular transformation.

A number of human malignancies are associated with aberrant or overexpression ofEGFR and/or overexpression of its specific ligands (e.g. transforming growth factor alpha). EGFR overexpression has been associated with an adverse prognosis in a number of human cancers, including non small cell lung cancer. In some instances, overexpression of tumor EGFR has been correlated with both chemoresistance and a poor prognosis. These observations suggest that agents that effectively inhibit EGFR receptor activation and subsequent downstream signaling may have clinical activity in a variety of human cancers.

Over the last several years inhibitors of these signal transduction pathways have been developed, taking advantage of specific sequential enzymatic steps that can be blocked selectively by targeted molecules. These inhibitors are generally of two classes 1) antibodies directed at the first step in the pathway, the cell surface receptor that interacts normally with the growth ligand, and 2) small molecules specifically designed to disrupt downstream enzymatic steps that lead from the cell surface to the nucleus where the growth signal is processed.

Growth factor pathway inhibitors have been in human clinical trials now for several years and most have an approximately 10% response rate with duration of responses lasting only a few months. These targeted therapies may be too “targeted” and many malignancies may require blocking a multitude of pathways to cause cell arrest and death. Combinations of growth factor inhibitors and chemotherapy show more promise with higher rates of response, 15-25% but still work for only a short period of time, usually only months. Tumor growth resumes shortly, despite the continued use of these agents, and shortly thereafter the host succumbs. More effective combination therapies are being sought, both to reduce dependence on chemotherapeutic agents because of their significant toxicities, as well as to increase rates and durability of response to growth factor blockade.

Currently there are two approved growth factor inhibitors in human solid malignancies: 1) Herceptin, a targeted antibody to Her2/neu, an essential growth pathway receptor in breast cancer and 2)IRESSA®(approved in Japan and Europe, and awaiting approval in the U.S.), a small molecule that inhibits the tyrosine kinase enzymatic portion of the EGF receptor (Her1), a pathway essential in lung, head and neck, colon and prostate cancer. Herceptin, has been shown to be clinically active as a single agent in breast cancer (16% response rates) as well in combination with chemotherapy (30-50%).IRESSA®, as a single agent in refractory/progressive lung cancer has overall rates of response in the range of 10-15%.

Retinoids

Retinol (vitamin A) and its natural and synthetic derivatives are known as retinoids. These signaling molecules are required for the proper finctioning of a large number of cell types with respect to processes such as cell differentiation and the control of cell growth arrest (reviewed in Gudas et al., 1994). Retinoids can also act as cancer preventive agents and are presently being used successfully to treat some types of cancer (reviewed in Moon et al., 1994; Hong et al., 1994; Warrell et al., 1991). The biological and transcriptional effects of retinoids are generally mediated by their interactions with the RARs and the RXRs (reviewed in Mangelsdorfet al., 1994 and Kastner et al., 1994). RARs and RXRs can heterodimerize and bind DNA response elements in a variety of target genes (reviewed in Refs. Gudas et al., 1994, Mangelsdorf et al., 1994 and Kastner etal., 1994).

The ligands for the RARs and RXRs have generally been thought to be acid derivatives ofretinol. RA binds to the RARs with high affinity, and 9-cis-RA binds and activates both the RXRs and the RARs (reviewed in Mangelsdorf et al., 1994 and Kastner et al., 1994). Retinol has been shown to be metabolized to RA in different types of cultured cells and in various tissues (Blaner et al., 1994 and Kurlandsky et al., 1994). However, the fact that retinol is metabolized into such a large number of different compounds (Randolph et al., 1993; Siegenthaler et al., 1990 and Torma et al., 1995), some of which have only recently been identified (Derguini et al., 1995; Buck et al., 1993; Derguini et al., 1994a; and Derguini et al., 1994b), implies that other metabolites of retinol may be biologically active with respect to receptor activation.

A novel retinoid synthetic pathway was identified in differentiating F9 embryonal mouse teratocarcinoma cells that results in the intracellular production of 4-oxo-retinol from retinol (vitamin A) (Achkar et al, 1996). Approximately, 10-15% of the total retinol in the culture is metabolized to 4-hydroxy-retinol and then to 4-oxo-retinol by the all-trans RA-treated, differentiating F9 cells over an 18 hour period, but no detectable metabolism of all-trans retinol to all-trans RA or 9-cis RA was observed in these cells (Achkar et al, 1996).

The retinoid receptor subtype-binding affinities of 4-oxo-retinol and 4-hydroxy-retinol were determined by competition with all-trans RA. Neither compound efficiently displaced all-trans RA, but measured by its ability to displace all-trans RA, 4-oxo-retinol specifically binds monomeric mouse RARa (IC₅₀ 330 nM) and b (IC₅₀ 420 nM) but not RARγ (IC₅₀ 3200 nM). These data are consistent with the interpretation that either 4-oxo-retinol is unable to bind RARY or that it binds RARγ at a different site than all-trans RA.

In contrast to its relative inability to displace all-trans-RA from its binding site on RAR, 4-oxo-retinol potently induced transactivation in human RARα-, but not RXRa-mediated trasactivation. The specificity of 4-oxo-retinol was further dissected utilizing chimeric reporter constructs in which the RARα-, β-, and α ligand binding domains were fused to a GAL4 DNA binding domain. In CV-1 cell transfectants, 4 oxo-retinol induces the highest activity on chimeras containing the ligand binding domain of RARg. In this system 4-oxo-retinol exhibits a greater ability to induce RARg activation than all-trans RA. Taken together with the inability of 4-oxo-retinol to displace all-trans RA, these data suggest that 4-oxo-retinol and all-trans RA bind RARγ at distinct sites, analogous to differences between 9-cis RA and all-trans RA binding sites on RARs (Levin et al, 1996).

Further studies addressed the biological roles of 4-oxo-retinol. In several biological systems, 4-oxo-retinol is similar to all-trans RA. For example, similar to all-trans RA, 4-oxo-retinol induces differentiation of F9 cells, transcription of the homeobox gene Hoxa-1 and laminin B1 synthesis. However, 4-oxo-retinol is substantially less teratogenic than all-trans RA in the Xenopus laevis system (ED₅₀ for 4-oxo-retinol˜300-400 nM vs. ED₅₀ for all-trans RA˜100 nM).

The production and metabolism of4-oxo-retinol has also been investigated. The half-life of 4-oxo-retinol (>15 hours) in F9 cells is much longer than for either retinol or all-trans RA, in part because 4-oxo-retinol is not converted to polar metabolites like RAs. The metabolism ofretinol was studied in normal human mammary epithelial cells as well as in a number of estrogen receptor-positive (ER+) and estrogen receptor-negative (ER-) human breast cancer cell lines (Chen et al. 1997). Although the numbers of cell lines analyzed remains small, it is strilcng that in the tumors examined to date, ER+ breast cancer cell lines MCF-7 and T47D produce 4-oxo-retinol from retinol, whereas ER- breast cancer cell lines MDA-MB-231, MDA-MB468 and BT20 do not.

Regardless ofthe correlation between ER expression, 4-oxo-retinol is growth inhibitory in all the ER+ or ER− tumor lines examined. Agents that augment 4oxo-retinol production (all-trans RA or N-(4-hydroxyphenyl)retinamide) are growth inhibitory in the tumors that are capable ofproducing it, but lack growth inhibitory effects on tumors that don't produce 4oxo-retinol. In summary, the analysis conducted thus far indicates strongly that 4-oxo-retinol possesses unique biological activities when compared with other active retinoids.

Vitamin D and Synergy with Other Retinoids

The molecular structure of the biological active form of vitamin D, 1α,25-10 dihydroxyvitamin D₃ (VD), and various vitamin A derivatives such as 4-oxo-retniol, all-trans and 9-cis retinoic acid (RA) are not related. The nuclear receptors for VD (VDR) and retinoids (RAR and RXR), however, are members of the same superfamily of ligand-activated transcription factors. Stable VDR-RXR and VDR-RAR heterodimers form in solution and have transcriptional activity on different types of response elements. Both heterodimeric complexes are activated by VD, but, depending on the relative expression of the nuclear receptors, retinoids can have either co-stimulating or repressing effects. This demonstrates that VD and retinoid signaling are linked at the level of gene regulation and may explain the similar effects of both hormones on cell proliferation and differentiation.

The formation of a heterodimer between two ligand-inducible nuclear hormone receptors bears the potential for transcriptional activation by two different ligands. Co-stimulation of VDR-RXR or VDR-RAR heterodimers with both VD and respective retinoids leads to additive or even synergistic effects of both specific ligands (Schrider et al, 1993, 1994, 1995). This indicates that heterodimerization not only increases the number of distinct nuclear receptor complexes, but also links the nuclear signaling pathways of different hormones.

Certain experimental observations indicate that such synergy occurs inbiological systems. For example, VD and all-trans RA cooperate in promoting the differentiation of HL60cells to monocytes in vitro (Brown et al, 1994). In another example, VD analogs and all-trans RA exhibit an additive or even synergistic activity in inhibiting cellular growth and DNA synthesis, and reduction of c-myc expression in HL60 cells (Dore et al, 1993). In yet another example, CBS-211A, a synthetic retinoid of low potency, alone does not affect proliferation and differentiation of U937 cells, but augments the activity of VD (Taimi et al, 1993). Finally, when administered systemically to mice, VD and either all-trans RA,13-cis RA, or 9-cis RA synergistically inhibited tumor cell-induced angiogenesis (Majewski et al, 1993).

There are also very preliminary clinical observations suggesting that some additive effect may occur in humans. Majewski et al (1994) observed responses of multiple actinic keratoses and squamous orbasal cell carcinomas infourpatients treated orally with both 13-cis RA and VD. French et al (1994) reported complete remission of cutaneous T-cell lymphoma in a single patient treated orally with the aromatic retinoid acitretin and the VD analog calcitriol (MC903). However, such synergistic effects are not always observed, particularly in vivo and even when they occur are not of great clinical significance.

### SUMMARY OF THE INVENTION

The present invention provides compositions comprising certain vitamin retinoid compounds which are useful for the treatment of various types of cancer and other disorders characterized by abnormal cell-proliferation and/or cell-differentiation. Optionally, the compositions of the present invention further comprise a growth factor pathway inhibitor and/or a vitamin D analog.

Specifically, the present invention provides a composition comprising a retinoid, wherein the retinoid is selected from the group of retinoid D with an alcohol CH₂OH terminal side chain, an ester of retinoid D having an ester bond, an ether of retinoid D having an ether bond, retinoid D where the alcohol CH₂OH terminal side chain is replaced with an aldehyde CHO terminal side chain, retinoid D with a carboxylic acid at the, terminal side chain wherein each of the ester bond and the ether bond is formed with the oxygen at the terminal side chain of Retinoid D and wherein retinoid D with the alcohol CH₂OH terminal side chain has the structure: 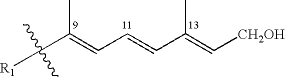
wherein the configuration at the 7-, 9-, 11- and 13-position double bonds is independently Z or E and wherein R. is selected from the group of 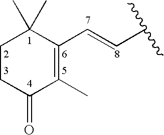
wherein the keto group at the 4-position is free or protected, or is replaced by a thioketone group which is free or protected or is replaced by C₁₋₆-alkylidene group; 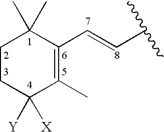
wherein X is selected from the group of hydrogen and C₁₋₆-alkyl and Y is selected from the group of C₁₋₆-alkyl, hydroxyl, C₁₋₆-alkoxyl, C₁₋₆-acyloxyl, halide, azide, sulfhydryl, amine and C₁₋₆-alkyl substituted amino and wherein the absolute configuration at the 4 position is independently R or S;

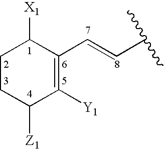

wherein X₁, Y₁ are independently selected from the group of hydrogen, C₁₋₆-alkyl, hydroxyl, C₁₋₆-alkoxyl, C₁₋₆-acyloxyl, halide, azide, sulfhydryl, amine and C₁₋₆-alkyl substituted amino and Z. is selected from the group of C₁₋₆-alkyl, hydroxyl, C₁₋₆-alkoxyl, C₁₋₆-acyloxyl, halide, azide, sulihydryl, amine and C₁₋₆-alkyl substituted amino; 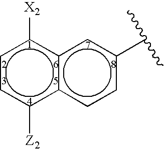
wherein X₂ is selected from the group of hydrogen, C₁₋₆-alkyl, hydroxyl, C₁₋₆-alkoxyl, C₁₋₆acyloxyl, halide, azide, sulfhydryl, amine and C₁₋₆-alkyl substituted amino and Z₂ is selected from the group of C₁₋₆-alkyl, hydroxyl, C₁₋₆-alkoxyl, C₁₋₆-acyloxyl, halide, azide, sulfhydryl, amine and C₁₋₆-alkyl substituted amino;

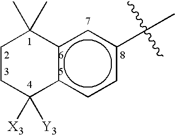

wherein X₃ and Y₃ are independently selected from the group of hydrogens, Cl 6-alkyl, hydroxyl, C,-alkoxyl, C,6-acyloxyl, halide, azide, sulfhydryl, amine and C₁xalkyl substituted amino so long as X₃ and Y₃ are not both hydrogens.

The present invention also provides that the retinoid binds and/or transactivates a Retinoic Acid Receptor or RXR

### DETAILED DESCRIPTION OF THE INVENTION

In accordance with the invention, it has been surprisingly discovered that a composition comprising only a certain type of retinoid is useful in treating a subject suffering from cancer or tumor growth as well as other disorders characterized by abnormal cell-proliferation and/or cell-differentiation.

It has also been discovered that addition of a growth factor receptor pathwayinhibitor and/or a vitaminD analog enhances the durabilityandrates ofresponse when treating cancer with the retinoids of the present invention.

A “tumor” is a neoplasm, and includes both solid and non-solid tumors (such as hematologic malignancies). A “hyperproliferative disease” is a disorder characterized by abnormal proliferation of cells, and generically includes slin disorders such as psoriasis as well as benign and malignant tumors of all organ systems. “Differentiation” refers to the process by which cells become more specialized to perform biological finctions, and differentiation is a property that is totally or partially lost by cells that have undergone malignant transformation.

A “therapeutically effective dose” is a dose which in susceptible subjects is sufficient to prevent advancement, or to cause regression of the disease, or which is capable of relieving symptoms caused by the disease, such as fever, pain, decreased appetite or chachexia associated with malignancy.

The cancer treated by the present invention is any and all types which includes and is not limited to melanoma, superficial squamous cell cancer of the skin, keratoacanthoma, head and neck cancers, thyroid cancer, lung both Non Small and Small cell lung cancer, thymoma, teratocarcinoma, hepatoma, gastric, brain, esophageal, pancreatic, cholangiocarcinoma, ampullary carcinoma, carcinoid, small bowel cancer, colon, appendiceal, rectal, anal, ovarian, breast, uterine or endometrial, fallopian tube, vaginal, cervical, penile, testicular, prostate, renal cell or kidney, lymphoma, acute leukemia, chronic lymphocytic leukemia, chronic myelogenous leukemia, osteosarcoma, sarcoma, glioma, multiple myeloma, astrocytoma, glioblastoma multiforme or eppendymoma.

For the purpose of this invention, treatment of a subject with the composition of the present invention means administering or applying to said subject either together or separately both the vitamin D analog and the retinoid as defmed in the composition of the present invention. Thus, the vitamin D analog and the retinoid may be administered or applied together or separately using either the same or different forms of administration or application.

For in vivo administration, administration is carried out by methods well known to those skilled in the art and include, but are not limited to, administration orally, parenterally including intravenously, and topically, and administration may be effected continuously or intermittently such that the amount of the composition in the patient is effective to obtain benefit.

The Retinoids

Specifically, the retinoids of the present invention are capable of binding and/or transactivating a Retinoic Acid Receptor (RAR) or RXR Examples of such retinoids which can be physiological or synthetic include but are not limited to retinoic acid, retinamide, bexarotene and tazarotene. When retinoic acid is the retinoid of choice, it can be any isomer including but not limited to all-trans-retinoic acid, 9-cis-rtinoic acid and 13-cis-retinoic acid.

Preferably, the retinoid can preferentially bind or transactivates an RAR or RXR selected from the group of: RARα:, RARβ RARγ , RXRα, RXRβ and RXRγ.

Also preferably, the retinoids of the present invention are is selected from the group of retinoid D with an alcohol CH₂OH terminal side chain, an ester of retinoid D having an ester bond, an ether of retinoid D having an ether bond, retinoid D where the alcohol CH₂OH terminal side chain is replaced with an aldehyde CHO terminal side chain, retinoid D with a carboxylic acid at the terminal side chain wherein each of the ester bond and the ether bond is formed with the oxygen at the terminal side chain of Retinoid D and wherein retinoid D with the alcohol CH₂OH terminal side chain has the structure:

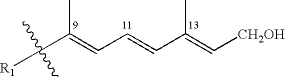

wherein the configuration at the 7-, 9-, 11- and 13-position double bonds is independently Z or E and wherein RI is selected from the group of 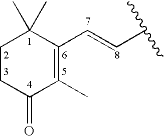
wherein the keto group at the 4-position is free or protected, or is replaced by a thioketone group which is free or protected or is replaced by C₁₋₆-alkylidene group; 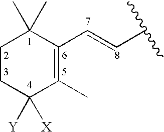
wherein X is selected from the group of hydrogen and C₁₋₆-alkyl and Y is selected from the group of C₁₋₆-alkyl, hydroxyl, C₁₋₆-alkoxyl, C₁₋₆-acyloxyl, halide, azide, sulfyhydryl, amine and C₁₋₆-alkyl substituted amino and wherein the absolute configuration at the 4 position is independently R or S; 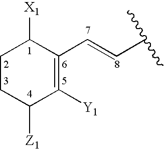
wherein X₁, Y₁, are independently selected from the group of hydrogen, C₁₋₆-alkyl, hydroxyl, C₁₋₆-alkoxyl, C₁₋₆-acyloxyl, halide, azide, sulfhydryl, amine and C₁₋₆-alkyl substituted amino and Z₁ is selected from the group of C₁₋₆alkyl, hydroxyl, C₁₋₆-alkoxyl, C₁₋₆-acyloxyl, halide, azide, sulfhydryl, amine and C₁₋₆-alkyl substituted amino; 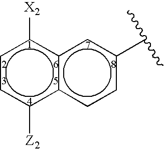
wherein X₂ is selected from the group of hydrogen, C₁₋₆-alkyl, hydroxyl, C₁₋₆-alkoxyl, C₁₋₆-acyloxyl, halide, azide, sulfhydryl, amine and C₁₋₆-alkyl substituted amino and Z₂ is selected from the group of C₁₋₆-alkyl, hydroxyl, C₁₋₆-alkoxyl, C₁₋₆-acyloxyl, halide, azide, sulfhydryl, amine and C₁₋₆-alkyl substituted amino; 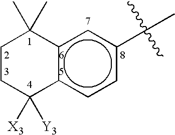
wherein X₃ and Y₃ are independently selected from the group of hydrogens, C₁₋₆-alkyl, hydroxyl, C₁₋₆-alkoxyl, C₁₋₆-acyloxyl, halide, azide, sulfhydryl, amine and C₁₋₆-alkyl substituted amino so long as X₃ and Y₃ are not both hydrogens.

The ester form contains ester group which is converted to alcohol in the body once adiministered or provides active compound without such conversion and said ester group normally contains from 1 to 16 carbon atoms if saturated, e.g., acetate or palmitate, orup to 20 carbon atoms if unsaturated, or is a modified sugar, e.g., a glucuronide. The ether form is the C₁₋₆-alkyl ether.

Retinyl esters of the retinoids of the present invention that are suitable for use in the present invention include but are not limited to C₁-C₃₀ esters of retinol, preferably C₂-C₂₀ esters, and most preferably C₂, C₃, and C₆ esters because they are commonly available. Examples of retinyl, esters include but are not limited to: retinyl palnitate, retinyl formate, retinyl acetate, retinyl propionate, retinyl butyrate, retinyl valerate, retinyl isovalerate, retinyl hexanoate, retinyl heptanoate, retinyl octanoate, retinyl nonanoate, retinyl decanoate, retinyl undecandate, retinyl laurate, retinyl tridecanoate, retinyl myristate, retinyl pentadecanoate, retinyl heptadecanoate, retinyl stearate, retinyl isostearate, retinyl nonadecanoate, retinyl arachidonate, retinyl behenate, retinyl linoleate, and retinyl oleate.

The preferred retinyl esters for use in the present invention are retinyl pairtate, retinyl acetate, retinyl propionate and retinyl linoleate. More preferred retinyl esters are S retinyl palmitate and retinyl acetate. the most preferred retinyl ester is retinyl palmitate.

Retinoid D is preferably 4-oxo-retinoic acid, 4-oxo-retinol, and 4-oxo-retinal, 4-hydroxy-retinol, 4-hydroxy-retinal, 4-oxo-retinyl ester, and 4-hydroxyretinyl ester. The most preferred retinoid is 4-oxo-retinol.

Pharmaceutically pure 4-oxo-retinol and 4-hydroxyretinol are readily obtained by inducing differentiation in F9 mouse teratocarcinoma stem cells, an established widely used line, from a murine tumor, available from the American Type Culture Collection under accession no. ATCC CRL 1720, with retinoic acid, whereby the endogenous retinol (vitamin A) is metabolized into many derivatives inthe differentiated cells, including 4-oxo-retinol and 4-hydroxyretinol, and isolating 4-oxo-retinol and 4-hydroxyretinol in pharmaceutically pure form. This can be carried out by culturing the F9 stem cells in the presence of retinoic acid (to induce differentiation) and retinol (to replace endogenous retinol as it is converted into derivatives) and extracting using the procedure of McClean, S. W., et al (1982) and isolating pharmaceutically pure 4-oxo-retinol and 4-hydroxyretinol from the extract using HPLC.

The compound 4-oxo-retinol can be prepared according to several known procedures as follows: A first method involves selective NaBH₄ reduction of 4-oxo-retinal, obtained by MnO.sub.2 oxidation of 4-hydroxy-retinal (See Boehm, M. F., et al, 1990). The latter can be prepared from commercially available all-trans-retinal (See Henbest, H. B., et al, 1957; Reedy, A. J., 1967; Surmatis, J. D. 1967; and Renk, G., et al, 1981). A second method involves the synthesis of 4-oxo-reti by direct oxidation of retinol or retinal by MnO₂ (See Henbest, H. B., et al; 1957 and Williams, T. C., et al, 1991). A third method involves hydrolysis of 4-oxo-retinyl acetate prepared from commercially available retinyl acetate (See Henbest, H. B., et al, 1957).

All-trans(4S) fhydroxy-retinol can be prepared starting with (4S)4hydroxy-.beta.-ionone obtained as described in Haag, A., et al (1980). Elongation of the side chain is achieved by conventional Homer-Emmons reactions as described in Haag, A., et al (1982) and Katsuta, Y., et al (1994) followed by HPLC purification.

All-trans(4R)4-hydroxy-retinol can be obtained similarly, starting with (4R)-4-hydroxy-.beta.-ionone obtained as described in Haag, A., et al (1980).

Racemic all-trans-4-hydroxy-retinol can be synthesized by NaBH₄ reduction of 4-hydroxy-, or 4-oxo-retinal obtained as described in Henbest, H. B., et al (1957); Reedy, A. J., et al, 1967); Surmatis, J. D., 1967); Renk, G., et al, 1981); and Williams, T. C., et al, 1991).

Others of the retinoids herein are prepared following retinoid synthetic procedures well known to those skilled in the art. See for example Dawson, M. I., et al (1990) which is incorporated herein by reference.

The retinoids herein are readily administered in vivo and optionally with other active ingredients as indicated above, as compositions comprising active compound in a pharmaceutically acceptable carrier. As used herein, the term “pharmaceutically acceptable carrier” encompasses any of the standard pharmaceutical carriers, including phosphate buffered saline solution, water, and emulsions such as an oil/water emulsion, and various types of wetting agents. In the preferred embodiment of the invention, the pharmaceutically acceptable carrier also comprises specific binding proteins, which may be, but are not limited to albumin, retinol binding proteins (RBP), transtlyretin (T-R), and the complex formed by RBP and TTR

The retinoids herein can be readily formulated with carrier for in vivo administration. Compositions for oral administration may be, for example, in capsule or pill form and comprise a therapeutically effective amount of retinoid herein and pharmaceutically acceptable excipient, e.g., inert diluent such as calcium carbonate, sodium carbonate, lactose or talc. Compositions for topical administration of retinoids herein may be, for example, in lotion, cream, ointment or gel form and comprise a therapeutically effective amount of retinoid herein and pharmaceutically acceptable carrier, e.g., propylene glycol, mineral oil, petrolatum, glyceryl monostearate and the like. Compositions for parenteral administration of retinoids herein include a therapeutically effective amount of retinoid herein and pharmaceutically acceptable carrier such as sterile water or physiological saline, and liposome delivery systems can be used to accommodate for lack of solubility.

In general, dosages for in vivo administration are 5 to 5000 mg ofretinoid herein per square meter of the body per day (0.01-100 mg/kg body weight/day), preferably 50 to 200 mg of retinoid herein per square meter of the body per day, for oral administration; 5 to 5000 mg of retinoid herein per square meter of the body per day, preferably 20 to 200 mg of retinoid herein per square meter of the body per day, for parenteral administration; and 1 mg to 500 mg of retinoid herein per square inch per day, preferably from 2 mg to 50 mg of retinoid herein per square inch per day, for topical administration.

More specifically, Bexarotene (targretin) dosage ranges from about 10 to lO0mg/m²/day, preferably about 100-400 mg/m²/day, more preferably about 400 mg/m²lday

Growth Factor Receptor Pathway lhhibitors

For the purpose of this invention, the term “growth factor receptor pathway inhibitor” is defined as any agent that is capable of either blocking the binding of a growth factor receptor to its natural ligand(s) or inhibiting growth factor receptor-mediated signaling. Thus, the term “growth factor receptor pathway inhibitor” includes and is not limited to antibodies to growth factor receptors as well as any agent or molecule that inhibits the transduction pathway of the growth factor. The antibody can be an animal, chimeric, human or humanized antibody.

Preferably, the growth factor receptor is an epidermal growth factor (EGF) receptor. Accordingly, the preferred “growth factor receptor pathway inhibitors is anti EGF receptor antibody (e.g.C225, a chimeric antibody), a fragment thereof, a single chain antibody, more preferably a monoclonal antibody and most preferably a humanized (human:murine chimeric antibody or humanized antibody) comprising a non-human variable and/or hypervariable region and a human constant region. The antibody may also be a full human antibody. The production of single chain, humanized and chimeric anti-EGF receptor antibodies is well known in the art (U.S. Pat. Nos. 5,558,864 and 5,844,093).

Alternatively, the agent used in the treatment can be any inhibitor of tyrosine kinase activity mediated by EGF receptors such as IRESSA® or any agent or compound that interferes with the binding of the EGF receptor to its natural ligands (i.e., EGF and TGF-alpha). For example, soluble forms of EGF receptors having the extracellular domain of EGF receptors could compete with EGF receptors for binding with EGF and TGF-alpha thus acting as inhibitors for the activation of EGF receptors.

Also preferably, the growth factor receptor is Her2/neu, another essential growth factor receptor. Accordingly, the preferred ” growth factor receptor pathway inhibitors is Herceptin, a targeted antibody to Her2/neu.

It is noteworthy that a person skilled in the art would understand that dosages and frequency of treatment vary depending on the tolerance of the individual patient and on the pharmacological and pharmacokinetic properties of each blocking or inhibitory agent used. Ideally, one wishes to achieve saturable pharmacokinetics for the agent used. When antibodies are used as blocking agents or inhibitors of the present invention, they can be administered to patients in any one of conventional ways well known to people skilled in the art For instance, antibodies such as C225 in a pharmaceutical composition can be administered intravenously. A loading dose (i.e., initial dose) can range for example, from about 10 to 1000 mg/r², preferably about 200 to 400 mg/m². This is followed by several additional daily or weekly dosages raging for example, from about 200 to 400 mg/m². The patient is closely monitored for side effects such as skin toxicity and the treatment is stopped when such side effects are severe.

Examples of growth factor inhibitors and/or other agents with preferred dosages that can be used on cancer patients in particular lung cancer patients most particular non small cell lung cancer include but are not limited to IRESSA® (gefitinib) (single agent dose of about 250 to 500 mg daily); IRESSA® (gefitinib) in combination with carboplatin (AUC 5-6 typical dose about 400-1000) plus paclitaxel (about 175-225 mg/m²) or gemcitabine (about 800-1250 mg/m²) plus cisplatin (75-100 mg/m²) or docetaxel (75 mg/m²); Erlotinib (OSI-774, TARCEVA®) 150 mg daily, cetuximab 400 mg/m2 IV on week one, then 250 mg/m2 weekly thereafter alone or in combination with docetaxel (75mg/m2) or paclitaxel (175-225 mg/m2) plus carboplatin (AUC 5-6 or typical doses of 400-1000 mg); Herceptin (trastuzumab) (4 mg/kg IV week one, followed by 2 mg/kg weekly thereafter) plus paclitaxel (about 225 mg/m2 every three weeks) and carboplatin (dosed at an area under the concentration X time curve [AUC]; Gleevec (imatinib) (300-600 mg, preferably 600 mg by mouth daily (small cell lung cancer); and Bryostatin which is a macrolide lactone isolated from the marine organism Bugula nerutina that inhibits the regulatory domain of PKC A.

Other agents that can be used in combination with the retinoids of the present invention include:
1) Antisense oligonucleotides to PKC : Affinitak (ISIS 3521, also known as LY90003);
2) Agents targeting the ras/MAPK pathway: The ras/MAPK pathway is implicated in cellular proliferation and inhibition of apoptosis. Inappropriate oncogenic activation of the MAPK pathway, such as by ras, is a feature of many neoplasms, including NSCLC. Examples of such agents include: antisense molecules (eg, ISIS 2503), farnesyl transferase inhibitors (FmIS) including tipifainib and lonafarnib; and
3)Raf kinase inhibitors: antisense molecules (eg, ISIS 5132), and the raf-1 linase inhibitor BAY 43-9006.
   Vitamin D Analogs

Also, for the purpose ofthis invention, the term “vitamin D analog” is defined as a compound capable of binding a vitamin D receptor or being converted in vivo into a compound capable of binding a vitamin D receptor. The term “vitamin D analog” includes but is not limited to vitamin D₂ and vitamin D₃ derivatives such as cholecalciferol, calcifediol, calcitriol, calcipotriol, ergosterol, ergocalciferol, dihydrotachysterol, 1,25-dihydroxyergocalciferol, 25-hydroxydihydrotachysterol, and the vitamin D analogs disclosed in U.S. Pat. No. 4,866,048. Preferred analogs are cholecalciferol, calcifediol, calcitriol, calcipotriol and the vitamin D analogs disclosed in U.S. Pat. No. 4,866,048. More preferred analogs are cholecalciferol, calcifediol, MC903, calcitriol and calcipotriol. Most preferred analogs are MC903, calcitriol and calcipotriol.

Preferably, the dose of vitamin D analog administered varies between about 0.01 to about 10 mcg per day, more preferably between about 0.25 to about 1.0 mcg per day.

Alternatively to daily dosing, vitamin D analogs may be administered to achieve intermittent supraphysiologic levels of 1,25-dihydroxyvitamin D (for example greater than or equal to 0.25 nM) which are also believed to be sufficient to inhibit cancer growth and other proliferative disease in marnmals. This approach permits the therapeutic benefits of Vitamin D therapy to be achieved without substantial risk of morbidity from iatrogenic hypercalcemia induced by the therapy.

Calcitriol is a short acting preparation of 1,25-dihydroxyvitamin D, which therefore offers an opportunity for intermittent treatment aimed at achieving high serum 1,25-dihydroxyvitamin D levels for brief periods of time. This regimen has surprising anti-tumor activity, while minimizing toxicity, such as hypercalcemia. Calcitriol has primarily been studied when chronically administered as replacement therapy, for which its usual dose is 0.25-1.0 mcg per day. Peak serum concentration is reached at 2 hours and serum half life is 3-6 hours. Intestinal absorption of calcium begins to increase 2 hours after administration. Hypercalcemic effect is maximal at 10 hours and lasts 3-5 days.

In one embodiment ofthe invention, a sufficient dose of calcitriol is administered to raise serum 1,25-hydroxyvitamin D levels to a therapeutically effective level for a pulsed dose that has an anti-proliferative effect without causing significant hypercalcemia (for example symptomatic grade 3 or grade 4 hypercalcemia). With 20 calcitriol, an example of such a dose would produce a serum level of at least about 0.5 nM, for example about 0.9 nM or more (e.g. 1-25 nM, for example 5-10 nM), for at least 2 hours (e.g. 2-5 hours) and preferably no more than 6 hours. In particular embodiments, the pulsed dose of calcitriol does not exceed a dose at which symptomatic hypercalcemia occurs, or more preferably a pulsed dose at which even laboratory hypercalcemia occurs.

A “pulse” dose of a Vitamin D analog refers to administration of the drug in a sufficient amount to increase the blood or tissue level of the Vitamin D drug to a supraphysiologic concentration for a sufficient period of time to have a therapeutic benefit, but with a sufficient period between doses to avoid hypercalcemia, given the pharmacological half life of the drug, its rate of elimination from the body, and its calcemic index.

Information about short term effects ofhigher than replacement doses ofcalcitriol is available for helping predict drug effects. Papapoulus et al., (1982) gave 2 mcg of calcitriol as a single oral dose to healthy volunteers and achieved peak 1,25-dihydroxyvitainin D serum concentrations of 0.235 and 0.351 nM. Mason et al. (1980) gave a single oral dose of 4 mcg calcitriol to healthy volunteers and achieved peak 1,25-dihydroxyvitamin D serum concentrations of 0.42 nM with no elevation in serum calcium. Briclinan et al. (1974) treated normal volunteers with calcitriol doses up to 2.7 mcg/day for 7 to 15 days. While calcium absorption and excretion were increased, no significant elevations in serum calcium were observed. Adams et al. (1982) treated normal volunteers with up to 3 mcgtday of calcitriol for 6-12 days and achieved stable 1,25-dihydroxyvitamin D serum levels of 0.184-0.235 nM. None of the patients who were on a low calcium diet experienced elevation in serum calcium. Geusens et al. (1991) gave 4 mcg of calcitriol per day for 4 days to 27 postmenapausal women with osteoporosis or osteoarthritis. They demonstrated increased urinary calcium excretion but no increase in urinary hydroxyproline excretion. Four of the 27 patients had a serum calcium above 10.8 but no clinically significant hypercalcemia was observed.

Antiproliferative levels of 1,25-dihydroxyvitamnin D can be achieved for short periods of time with minimal adverse effects, particularly if hypercalcemia during short course 1,25-dihydroxyvitamin D therapy is primarily mediated by increases in intestinal calcium absorption (slower calcium elevation) rather then osteoclast activation (which can rapidly mobilize calcium from bone). Higher 1,25-dihydroxyvitamin D levels are achievable when the drug is given in conjunction with a reduced calcium diet to minimize intestinal calcium absorption, and adequate hydration to maximize calcium excretion. The maximal tolerated dose of calcitriol, when given intermittently has not been defined, but doses as high as 0.48 mcg/kg have been tolerated without hypercalcemia.

Higher doses of a Vitamin D drug, sufficient to achieve therapeutic antiproliferative levels, may also be achieved by administering the drug in conjunction with bisphosphonate osteoclast inhibitors, such as pamidronate. Selby et al. (1981) provided an example of treating hypercalcemia due to Vitamin D with pamidronate. The potential for achieving high serum 1 ,25-dihydroxyvitamin D levels when osteoclasts are inhibited in patients with osteopetrosis is possible with calcitriol doses as high as 32 mcg/day for 3 months (Key et al., 1984) where stable serum levels of 1,25-dihydroxyvitamin D peaked at 2.32 nM with no hypercalcemia.

For the purpose of this invention, treatment of a subject with the composition of the present invention means administering or applying to said subject either together or separately any and all of the retinoid defined above, optionally the growth factor receptor pathway inhibitor and frrther optionally the vitamin D analog. Thus, each of these agents can be administered alone or together or separately by different means or forms of administration or application.

Other Treatments

Irradiation therapy can be conducted and/or a pharmaceutically effective dose of at least one chemotherapy agent can be administered before, during and/or after the composition(s) of the present invention. Also preferably, the chemotherapy agent is selected from the group of IRITONECAN® (CPT-11), 5-florouracil (5-FU), CISPLATIN®(CDDP), OXALOPLATIN®, LEUCOVORIN® and BRYOSTATIN®, most preferably IRITONECAN®.

As indicated above, radiation or chemotherapy treatment may be employed before, during or after the use of the retinoids and/or blocking agents or inhibitors of the present invention. Protocols using numerous irradiation treatments and/or chemotherapy agents are well known in the art (Rothenberg et al., J Clin. Onc., 14(4): 1128-1135, 1996). Preferably, the chemotherapy agent(s) used is the one that normally is the most effective for the particular case. For example, IRITONECAN® is a preferred agent since it was found to have significant single-agent activity against colorectal cancer (Rothenberg et al., 1996).

Chemotherapy Treatment of NSCLC (non small cell lung cancer) include but are not limited to the following: cisplatin (75-100 mg/m²) plus etoposide (100 mg/m²) plus ifosfamide (2 g/m²); mitomycin (10 mg/m2) plus ifosfamide (2 g/m²) plus cisplatin (75-100 mg/m²) a; cisplatin (75-100 mg/m) plus paclitaxel (as a 24 hour infusion at one of two different doses, 135 mg/m² or 250 mg/m²); cisplatin (80 mg/m² every 3 weeks) plus paclitaxel (175-225 mg/m² over 3 hours every 3 weeks); carboplatin (AUC 5-6 typical mg dose is 400-1200 mg) plus paclitaxel (175-225 mg/m²); gemcitabine (800-1000 mg/m² on days 1 and 8) plus paclitaxel (175-225 mg/m2 over 3 hours on day 1, both administered every three weeks); irinotecan (125 mg/m²/week); docetaxel (100 mg/mn² on day 1) plus cisplatin (80 mg/m² on day 2); gemcitabine (800- 1200 mg/m² days 1 and 8) plus cisplatin (80 mg/m² on day 1); gemcitabine (800-1200 mg/m² day 1 and 8) plus carboplatin (AUC 5 on day 1); gemcitabine (1000 mg/I² on days 1 and 8) plus docetaxel (100 mg/m² on day one); gemcitabine (1000 mg/m² on days 1 and 8) plus vinorelbine (25 mg/m² days 1 and 8); vinorelbine (25-30 mg/m² on days 1 and 8 of every 21 day cycle); vinorelbine (25-30 mg/m² days 1 and 8) plus cisplatin (80-100 mg/m² on day 1); paclitaxel (90 mg/m2 weekly); Docetaxel (75-100 mg/m² every three weeks);

Gemcitabine (800 to 1750 mg/m² per week for three of every four weeks); paclitaxel (225 mg/m² over three hours); docetaxel (36 mg/m² weekly for six of every eight weeks); docetaxel (100 or 75 mg/m² once every three weeks); ifosfamide (2 g/m² daily for three days, every three weeks); pemetrexed 500 mg/mn² (supplemented with folic acid, vitamin B12 and dexamethasone); cisplatin (100 mg/m² on day 1) plus vindesine (3 mg/m²); cyclophosphamide plus doxorubicin and cisplatin (CAP); cisplatin (75-100 mg/m²) plus vinblastine (2-4 mg/m²); cisplatin (120 mg/m) day 1 plus etoposide (100 mg/m² IV on days 1 to 3); mitomycin (8-10 mg/m²) plus vindesine (3 mg/m2), and cisplatin (75-100 mg/m²); ifosfamide (2 g/m²) plus cisplatin (75-100 mg/m²); cisplatin (75-100 mg/m²) plus etoposide (100 mg/m²) plus ifosfamide (2 g/m²); and mitomycin (10 mg/m²) plus ifosfamide (2 g/m²) plus cisplatin (75-100 mg/m²).

### EXAMPLE 1

A 39 year old male was diagnosed with acute lymphoblastic leukemia. He 15 received a chemotherapy induction regimen of eight cycles of Hyper-CVAD which consists of the following: Cyclophosphamide 300 mg/mn² intravenously (IV) over 3 hours every 12 hours for six doses on days 1 through 3, with mesna at the same total dose as cyclophosphamide but given bycontinuous infusion starting with cyclophosphamide and ending 6 hours after the last dose; vincristine 2 mg IV days 4 and 11; doxorubicin 50 mg/² IV day 4; and dexamethasone 40 mg daily on days 1 through 4 and 11 through 14.

In addition, high dose methotrexate-cytarabine (ara-c) was used as follows: MTX (methotrexate) 200 mg/m² IV over 2 hours followed by 800 mg/m² IV over 24 hours on day 1; citrovorum factor rescue starting 24 hours after completion of MTX infusion at 15 mg every 6 hours x 8, and increased to 50 mg every 6 hours if MTX levels were more 25 than 20 ,upmol/L at the end of the infuision, more than 1 μμmol/L 24 hours later, or more than 0.1 μμmol/L48 hours after the end of MTX infusion, until levels were lower than 0.1μμM; ara-C (cytarabine) g/m² over 2 hours every 12 hours ×4 on days 2 and 3; and methylprednisolone 50 mg IV twice daily on days 1 through 3.

Following the induction regimen, the patient went into a complete remission. About a year later, he relapsed with the presence of cancer cells in blood and bone marrow. Thereafter, he underwent a salvage chemotherapy regimen of FLAG (fludarabine, ara-c and G-CSF) with the following doses: fludarabine 30 mg/m²lday for 5 days; ara-C (cytarabine) 2 g/m²/day for 5 days (FLAG); and G-CSF (neupogen or granulocyte colony stimulating factor) 400 micrograms/m²/day. The patient did not respond to the salvage treatment and started showing enlarged lymph nodes. He was then given the same salvage regimen plus a daily oral dose of 4-oxo-retinol at 150 mg per day for 28 days. The patient responded with a complete remission (in blood and bone marrow) and shrinkage of the lumph nodes to normal size. This remission lasted for one month before relapse.

The same salvage regimen was repeated with oral administration of4-oxo-retinol (75 mg per day for 18 days) in addition to a daily dose of calcitriol (50 mcg per day concomitant with the administration of 4-oxo-retinol). The patient once again went into complete remission and was being prepared for a bone marrow transplant.

### EXAMPLE 2

A patient suffering from non small cell lung cancer was treated with IRESSA® (250 mg per day) for two months. The disease progressed during the treatment and thae patient was continued on IRESSA® (250 mg per day) with daily dosage of 4-oxo-retinol (75 mg per day) for two months. The disease became stable and the patient continued taking IRESSA® (250 mg per day) for 4 more months without 4-oxo-retinol with a stale disease.

### EXAMPLE 3

Various types of human melanoma, breast cancer and prostate cancer cells will be cultured according to standardized procedures. These cells will be incubated with in the presence of various concentrations of 4-oxo-retinol, growth factor receptor inhibitor (EGF receptor antibody or tyrosine kinase inhibitor), calcitriol or a combination thereof.

Cell growth of at least some of these cells will be shown to be significantly inhibited in the presence of4-oxo-retinol and growth factor receptor inhibitor as compared with cells incubated in absence of the above compounds or in the presence of each of the above compounds alone.

### EXAMPLE 4

Patients with breast cancer, prostate cancer or leukemia, particularly acute promyelocytic leukemia treated with a combination of oral doses of 4-oxo-retinol or 4-hydroxy-retinol (100mg/square meter) and oral doses of IRESSA® will have a reduced tumor burden as compared with patients treated with IRESSA® alone and will undergo prolonged remission or are permanently cured.

### EXAMPLE 5

Current systemic chemotherapy regimens are unable to prolong survival of patients with advanced head and neck cancer. Patients treated with a combination of oral doses of 4-oxo-retinol or 4-hydroxy-retinol (lOOmg/square meter) and oral doses of IRESSA® or intravenous doses of C-225 will have a better survival rate as compared with patients treated with IRESSA® or C-225 alone, and/or will have reduced tumor burden during the period during which this treatment is administered.

### EXAMPLE 6

Patients with lung cancer treated with a combination of oral doses of 4oxo-retinolor4-hydroxy-retinol (100mg/squaremeter) and oral doses ofIRESSA® will have a reduced tumor burden as compared with patients treated with IRESSA® alone and will undergo prolonged remission or are permanently cured.

### EXAMPLE 7

Patients with lung cancer who have shown disease progression upon treatment with IRESSA® alone will have a reduced tumor burden and will undergo prolonged remission or are permanently cured when their IRESSA® treatment is supplemented with oral doses of 4-oxo-retinol or 4-hydroxy-retinol (100mg/square meter) and optionally oral doses of calcitriol (0.1-1 mcg per day).

### EXAMPLE 8

Patients with small cell lung cancer who have shown disease progression upon treatment with IRESSA® (about 250-500 mg per day)alone will have a stable disease or a reduced tumor burden and will undergo prolonged remission or are permanently cured when their IRESSA® treatment is supplemented with oral doses of bexarotene (about 100 to 500 mg per day, preferably about 400 mg per day).

The invention has been described in terms ofpreferred embodiments thereof, but is more broadly applicable as will be understood by those skilled in the art. The scope of the invention is therefore limited only by the following claims.

REFERENCES Achkar, C. C., Derguini, F., Blumberg, B., Langston, A., Levin, A. A., Speck, J., Evans, R. M., Bolado, J., Jr., Nakanishi, K-, Buck, J., and.Gudas, L. J. 4-Oxoretinol, a new natural ligand and tansactivator of the retinoic acid receptors. Proc. Natl. Acad. Sci. USA, 93: 4879-4884, 1996.

Adams et al. Kidney Int. 21:90-97, 1982.

Boehm, M. F., Gawinowicz, M. A., Foucault, A., Derguini, F., and Nakanishi, K. (1990) Photoaffinity labeling studies of bacteriorhodopsin with [15-3 H]-3-diazo-4-keto-all-trans-retinal. J. Am. Chem. Soc. 112: 7779-7782.

Brown, G, Bunce CM, Rowlands DC, Williams GR: All-trans retinoic acid and 1,a25-dihydroxyvitamin D₃ co-operate to promote differentiation of the human promyeloid leukemia cell line HL60to monocyte. Leukemia 8:806-815, 1994.

Blaner, W. S., and Olson, J. A. Retinol and retinoic acid metabolism. In: M. B. Spom, A. B. Roberts, and D. S. Goodman (eds.), The Retinoids: Biology, Chemistry, and Medicine, pp. 229-256. New York: Raven Press, 1994.

Brickinan et al. Am. J. Med. 57:28-33, 1974

Buck, J., Grim, F., Kimura, S., Noy, N., Derguini, F., and Hammerling, U. Anhy-droretinol: a naturally occurring inhibitor of lymphocyte physiology. J. Exp. Med., 178: 675-680, 1993.

Chen C, Guo X, Derguini F, and Gudas L: Human breast cancer cells and normal mammary epithelial cells: Retinol metabolism and growth inhibition by the retinol metabolite 4-oxoretinol. Cancer Res. 57:46424651, 1997.

Dawson, M. I., et al, Editors, Chemistry and Biology of Synthetic Retinoids, CRC Press Inc., 1990.

Derguini, F., Nakanishi, K, Hammerling, U., and Buck, J. Intracellular signaling activity of synthetic (14R)-, (14S)-, and (14RS)-14-hydroxy4,14-retro-retinol. Biochemistry, 33: 623-628, 1994b.

Derguini, F., Nakanishi, K., Buck, J., Hammerling, U., and Griin, F. Spectroscopic studies of anhydroretinol, an endogenous mammalian and insect retro-retinol. Angew. Chem. Int. Ed. Engl., 33: 1837-1839, 1994b.

DoréB T, Uskokovic M R, Momparler R L: Interaction of retinoic acid and vitamin D₃ analogs on HL-60 myeloid leukemic cells. LeukRes 17:749-757, 1993.

French L, Ramelet A A, Saurat J-H: Remission of cutaneous T-cell lymphoma with combined calcitriol and acitretin. Lancet 344:686-687, 1994.

Geusens et al. Calcifed Tissue Int. 49:168-173, 1991.

Gudas, L. J., Spom, M. B., and Roberts, A. B. Cellular biology and biochemistry of the retinoids. In: M. B. Sporn, A. B. Roberts, and D. S. Goodman (eds.), The Retinoids: Biology, Chemistry, and Medicine, pp. 443-520. New York: Raven Press, 1994.

Haag, A. and Eugster, C. H. (1980) Synthesevon(-)-(R)-4-Hydroxy-.beta.-iononund(-)-(5S, 6S) -5-Hydroxy4,5-dihydro-.alpha.-ionon aus(-)-S-.alpha.-Ionon. Helv. ChenL Acta 63:10-15.

Haag, A. and Eugster, C. H. (1982) Isozeaxanthine: Chiralitat und enantioselective Synthese von(4R,4R′) -Isozeaxanthin,)(-)-(4R,4R′)-.beta.,.beta.-Carotin-4,4′diol!. Helv. Chim. Acta 65: 1795-1803.

Henbest, H. B., Jones, E., and Owen, T. C. (1957) Studies in the polyene Series. Part LII. Oxidation of vitamin A and retinene by manganse dioxide. J. Chem. Soc. 4909-4912.

Hong, W. K., and Itri, L. M. Retinoids and human cancer. In: M. B. Spom, A. B. Roberts, and D. S. Goodman (eds.), The Retinoids: Biology, Chemistry, and Medicine, pp. 597-630. New York: Raven Press, 1994.

Kastner, P., Leid, M., and Chambon, P. The role of nuclear retinoic acid receptors in the regulation of gene expression. In: R. Blomhoff (ed.), Vitamin A in Health and Disease, pp. 189-238. New York: Marcel Dekker, Inc., 1994.

Katsuta, Y., Yoshihara, K., Nakanishi, K, and Ito, M. (1994) Synthesis of (+)-(4S)- and (-)-(4R)- 1 1ZAhydroxyretinals and determination of the absolute stereochemistry of a visual pigment chromophore in the bioluminescent squid, Watasenia scintillans, Tetrahedron Lett. 35: 905-908.

Key et al., NEJM 310:409415, 1984.

Kurlandsky, S. B., Xiao, J. H., Duell, E. A., Voorhees, J. J., and Fisher, G. J. Biological activity of all-trans retinol requires metabolic conversion to all-trans retinoic acid and is mediated through activation ofnuclear retinoid receptors inhuman keratinocytes. J. Biol. Chem., 269: 32821-32827, 1994.

Levin A, Struzenbecker L, Kazmer S: 9-Cis retinoic acid stereoisomer binds and activates thenuclearreceptorRXR6. Proc. Natl. Acad. Sci. USA. 93:4879-4884,1996).

Mason et al. BMJ 449-450, 1980.

McClean, S. W., Ruddel, M. E., Gross, E. G., Degiovanna, J., and Peck, G. L. (1982) Liquid chromatographic assay for retinol (vitamin A) and retinol analogs in therapeutic trials. Clin Chem. 284: 693-696.

Majewski S, Skopinska M, Bollag W, Jablonska S: Combination of isotretinoin and calcitriol for precancerous and cancerous skin lesions. Lancet 344:1510-1511, 1994.

Majewski S, Szmurlo A, Marczak M, Jablonska S, Bollag W: Inhibition of tumor cell-induced angiogenesis by retinoids, 1,25-dihydroxyvitamin D₃ and their combination. Cancer Lett 75:35-39, 1993.

Mangelsdorf, D. J., Umesono, K., and Evans, R. M. The refinoid receptors. In: M. B. Spom, A. B. Roberts, and D. S. Goodman (eds.), The Retinoids: Biology, Chemistry, and Medicine, pp. 319-350. New York: Raven Press, 1994.

Moon, R. C., Mehta, R. G., and Rao, K V. N. Retinoids and cancer in experimental animals. In: M. B. Sporn, A. B. Roberts, and D. S. Goodman (eds.), The Retinoids: Biology, Chemistry and Medicine, pp. 573-596. New York: Raven Press, 1994.

Papapoulus et al., Clinical Science 62:427429, 1982.

Randolph, R. K, and Simon, M. Characterization of retinol metabolism in cultured human epidermal keratinocytes. J. Biol. Chem., 268: 9198-9205, 1993.

Reedy, A. J. (Jun. 9, 1967) Procede de synthese du 4hydroxy-retinal. Fr. Patent 1,484, 573. Chem. Abstr. (1968) 68: 29903m.

Renk, G., Grover, T., Crouch, R., Mao, B., and Ebrey, T. (1981) A spin labeled retinal pigment analogue of the purple membrane. Photochem. Photobiol. 33: 489494.

Rothenberg et al., J Clin. Onc., 14(4):1128-1135, 1996.

Schräder M, Bendik I, Becker-AndréM, Carlberg C: Interaction between retinoic acid and vitamin D signalling pathways. JBiol Chem 268:17830-17836, 1993.

Schräder M, Müller KM, Becker-Andre M, Carlberg C: Response element selectivity for heterodimerization of vitamin D receptors with retinoic acid and retinoid X receptors. J Moi Endocrinol 12:327-339, 1994. Schräder M, Nayeri S, Kahlen J-P, Müller K M, Carlberg C: Natural vitamin D₃ response elements formed by inverted palindromes: polarity-directed ligand sensitivity of VDR-RXR heterodimer-mediated transactivation. Mol Cell Biol 15:1154-1161, 1995.

Siegenthaler, G., Saurat, J-H., and Ponec, M. Retinol and retinal metabolism: relationship to the state of differentiation of cultured human keratinocytes. Biochem.. J., 268:371-378, 1990.

Selby et al. Endocrinology 108:1083-1086, 1981.

Surmatis, J. D. (Mar. 28, 1967) Intermediates for the preparation of a carotenoid. U.S. Pat. No. 3,311,656. Chem. Abstr. (1967) 67: 22052d.

Taimi M, Defacque H, Commes T, Favero J, Dornand J, Marti J: The retinoic acid analog CBS-211A potentiates the 1α,25-dihydroxyvitamin D₃-induced differentiation of U937 cells. Agents Actions 38:91-99, 1993.

Torma, H., and Vahlquist, A. Vitamin A esterification inhuman epidermis: a relationship to keratinocyte differentiation. J. Invest. Dermatol., 94: 132-138, 1990.39. Derguini, F., Nakanishi, K., Hammerling, U., Chua, R., Eppinger, T., Levi, E., and Buck, J. 13,14-Dihydroxy-retinol, a new bioactive retinol metabolite. J. Biol. Chem., 270:19975-18880, 1995.

Warrell, R. P., Jr., de The, H., Wang, Z-Y., and Degos, L. Acute promyelocytic leukemia. N. Engl. J. Med., 329: 177-189, 1991.

Williams, T. C. and Mani, V. (1991) Design ofa helix-bundle cross-link: NMR and UV-visible spectroscopic analysis and molecular modeling of ring-oxidized retinals. Biochemistry 30: 2976-2988.

What is claimed is:

1. A method of treating a subject suffering from a disorder characterized by abnormal cell-proliferation and/or cell-differentiation, comprising administering to the subject in need of such treatment a pharmaceutically effective dose of a growth factor receptor inhibitor and a retinoid wherein:
the retinoid is selected from the group of retinoid D with an alcohol CH₂OH terminal side chain, an ester of retinoid D having an ester bond, an ether of retinoid D having an ether bond, retinoid D where the alcohol CH₂OH terminal side chain is replaced with an aldehyde CHO terminal side chain, retinoid D with a carboxylic acid at the terminal side chain wherein each of the ester bond and the ether bond is formed with the oxygen at the terminal side chain of Retinoid D and wherein retinoid D with the alcohol CH₂OH terminal side chain has the structure: 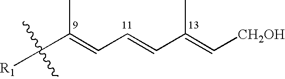
wherein the configuration at the 7-, 9-, 11- and 13-position double bonds is independently Z or E and wherein RI is selected from the group of 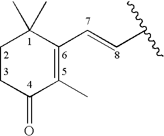
wherein the keto group at the 4-position is free or protected, or is replaced by a thioketone group which is free or protected or is replaced by C₁₋₆-alkylidene group; 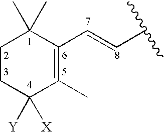
wherein X is selected from the group of hydrogen and C₁₋₆-alkyl and Y is selected from the group of C₁₋₆-alkyl, hydroxyl, C₁₋₆-alkoxyl, C₁₋₆-acyloxyl, halide, azide, sulfhydryl, amine and C₁₋₆-alkyl substituted amino and wherein the absolute configuration at the 4-position is independently R or S; 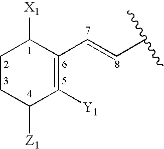
wherein X₁, Y₁, are independently selected from the group of hydrogen, C₁₋₆-alkyl, hydroxyl, C₁₋₆-alkoxyl, C₁₋₆-acyloxyl, halide, azide, sulflhydryl, amine and C₁₋₆-alkyl substituted amino and Z₁ is selected from the group of C₁₋₆-alkyl, hydroxyl, C₁₋₆-alkoxyl, C₁₋₆-acyloxyl, halide, azide, sulffiydryl, amine and C₁₋₆-alkyl substituted amino; 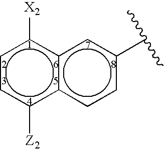
wherein X₂ is selected from the group of hydrogen, C₁₋₆-alkyl, hydroxyl, C_{1-6-alkoxyl, C}₁₋₆-acyloxyl, halide, azide, sulfhydryl, amine and C₁₋₆-alkyl substituted amino and Z₂ is selected from the group of C₁₋₆-alkyl, hydroxyl, C₁₋₆-alkoxyl, C₁₋₆-acyloxyl, halide, azide, sulfhydryl, amine and C₁₋₆-alkyl substituted amino; 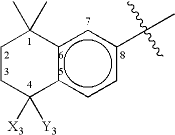
wherein X₃ and Y₃ are independently selected from the group of hydrogens, C₁₋₆-alkyl, hydroxyl, C₁₋₆-alloxyl, C₁₋₆-acyloxyl, halide, azide, sulfhydryl, amine and C₁₋₆-alkyl substituted amino so long as X₃ and Y₃ are not both hydrogens.

2. The method of claim 1, wherein the retinoid retinoid D with the alcohol CH₂OH terminal side chain has the structure: 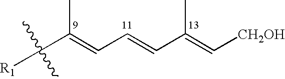
wherein the configuration at the 7-, 9-, 11- and 13-position double bonds is independently Z or E and wherein R₁ is selected from the group of:
wherein the keto group at the 4position is free or protected; and 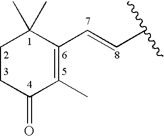 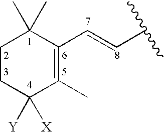
wherein X is selected from the group of hydrogen and C₁₋₆-alkyl and Y is selected from the group of hydroxy and C₁₋₆-alkyoxyl and wherein the absolute configuration at the 4-position is independently R or S.

3. The method of claim 1, wherein the retinoid is retinoid D with an alcohol CH₂OH terminal side chain.

4. The method of claim 1, wherein the retinoid is selected from the group of 4-oxo-retinol, 4-oxo-retinoic acid, 4-oxo-retinal, and 4-oxo-retinyl ester.

5. The method of claim 1, wherein the retinoid is all-trans 4-oxo-retinol or an isomer thereof.

6. The method of claim 1, wherein the retinoid is 4-hydroxy-retinol.

7. The method of claim 1, wherein the retinoid is 4methoxy-retinol.

8. The method of claim 1, wherein said growth factor receptor is EGF receptor.

9. The method of claim 8, wherein said EGF receptor inhibitor is an EGF receptor antibody.

10. The method of claim 9, wherein said antibody is C-225.

11. The method of claim 8, wherein said EGF receptor inhibitor is an inhibitor of tyrosine kinase activity mediated by EGF receptors.

12. The method of claim 11, wherein said EGF receptor inhibitor is IRESSA®

13. The method of claim 1, wherein said growth factor receptor is TGF-alpha.

14. The method of claim 1 further comprising administering to the subject a vitamin D analog.

15. The method of claim 14, wherein the vitamin D analog is selected from the group of cholecalciferol, calcifediol, calcitriol, calcipotriol, ergocalciferol, dihydrotachysterol, 1,25-dihydroxyergocalciferol, and 25-hydroxydihydrotachysterol.

16. The method of claim 15, wherein the vitamin D analog is calcitriol.

17. The method of claim 15, wherein the vitamin D analog is calcipotriol.

18. The method of claim 1 further comprising administering to said subject at least one chemotherapy agent.

19. The method of claim 1 further comprising treating said subject with irradiation.

20. The method of claim 1, wherein said disorder is cancer.

21. The method of claim 20, wherein said cancer is selected from the group of: melanoma, superficial squamous cell cancer of the skin, keratoacanthoma, head and neck cancers, thyroid cancer, lung both Non Small and Small cell lung cancer, thymoma, teratocarcinoma, hepatoma, gastric, brain, esophageal, pancreatic, cholangiocarcinoma, ampullary carcinoma, carcinoid, small bowel cancer, colon, appendiceal, rectal, anal, ovarian, breast, uterine or endometrial, fallopian tube, vaginal cervical, penile, testicular, prostate, renal cell or kidney, lymphoma, acute leukemia, chronic lymphocytic leukemia, chronic myelogenous leukemia, osteosarcoma, sarcoma, glioma, astrocytoma, multiple myeloma, glioblastoma multiforme and eppendymoma.

22. A method of treating a subject suffering from a disorder characterized by abnormal cell-proliferation and/or cell-differentiation, comprising administering to the subject in need of such treatment a pharmaceutically effective dose of a growth factor receptor inhibitor and a retinoid that binds and/or transactivates a Retinoic Acid Receptor or RXR.

23. The method of claim 22, wherein the retinoid is selected from the group of retinoic acid, retinamide, bexarotene and tazarotene.

24. The method of claim 22, wherein the Retinoid Acid receptor (RAR) is selected from the group of: RARα, RARβ and RARγ.

25. The method of claim 22, wherein the RXR is selected from the group of: RXRα, RXRβ and RXRγ.

26. The method of claim 23, wherein the retinoic acid is selected from the group of isomers of: all-trans-retinoic acid, 9-cis-rtinoic acid and 13-cis-retinoic acid.

27. The method of claim 23, wherein the retinoid is bexarotene, the disorder is non small cell lung cancer and the growth factor receptor inhibitor is IRESSA®.

28. A method of treating a subject suffering from cancer, comprising administering to the subject in need of such treatment a pharmaceutically effective dose of a retinoid wherein:
the retinoid is selected from the group of retinoid D with an alcohol CH₂OH terminal side chain, an ester of retinoid D having an ester bond, an ether of retinoid D having an ether bond, retinoid D where the alcohol CH₂OH terminal side chain is replaced with an aldehyde CHO terminal side chain, retinoid D with a carboxylic acid at the terminal side chain wherein each of the ester bond and the ether bond is formed with the oxygen at the terminal side chain of Retinoid D and wherein retinoid D with the alcohol CH₂OH terminal side chain has the structure: 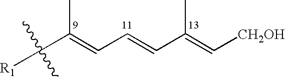
wherein the configuration at the 7-, 9-, 11- and 13-position double bonds is independently Z or E and wherein R₁ is selected from the group of 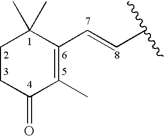
wherein the keto group at the 4position is free or protected, or is replaced by a thioketone group which is free or protected or is replaced by C₁₋₆-alkylidene group; 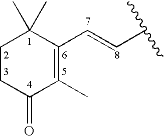
wherein X is selected from the group of hydrogen and C₁₋₆-alkyl and Y is selected from the group of C₁₋₆-alkyl, hydroxyl, C₁₋₆-alkoxyl, C₁₋₆acyloxyl, halide, azide, sulfhydryl, amine and C₁₋₆-alkyl substituted amino and wherein the absolute configuration at the 4-position is independently R or S; 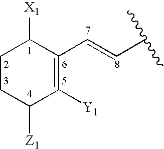
wherein X₁, Y₁, are independently selected from the group of hydrogen, C₁₋₆-alkyl, hydroxyl, C₁₋₆-6-alkoxyl, C₁₋₆-6-acyloxyl, halide, azide, sulfhydryl, amine and C₁₋₆-6-alkyl substituted amino and Z₁ is selected from the group of C₁₋₆alkyl, hydroxyl, C₁₋₆4-alkoxyl, C₁₋₆-acyloxyl, halide, azide, sulfhydryl, amine and C₁₋₆-alkyl substituted amino; 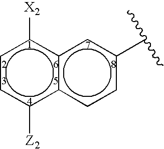
wherein X₂ is selected from the group of hydrogen, C₁₋₆-alkyl, hydroxyl, C₁₋₆-alkoxyl, C₁₋₆-acyloxyl, halide, azide, sulfhydryl, amine and C₁₋₆-alkyl substituted amino and Z2 is selected from the group of C₁₋₆-alkyl, hydroxyl, C₁₋₆-alkoxyl, C₁₋₆-acyloxyl, halide, azide, sulfhydryl, amine and C₁₋₆-alkyl substituted amino; 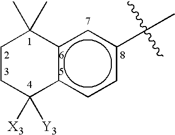
wherein X₃ and Y₃ are independently selected from the group of hydrogens, C₁₋₆-alkyl, hydroxyl, C₁₋₆-alkoxyl, C₁₋₆₄-acyloxyl, halide, azide, sulihydryl, amine and C₁₋₆-alkyl substituted amino so long as X₃ and Y₃ are not both hydrogens; and
wherein said cancer is selected from the group: melanoma, superficial squamous cell cancer of the skin, keratoacanthoma, head and neck cancers, thyroid cancer, lung both Non Small and Small cell lung cancer, thymoma, teratocarcinoma, hepatoma, gastric, brain, esophageal, pancreatic, cholangiocarcinoma, ampullary carcinoma, carcinoid, small bowel cancer, colon, appendiceal, rectal, anal, ovarian, uterine or endometrial, fallopian tube, vaginal, cervical, penile, testicular, prostate, renal cell or kidney, lymphoma, acute lymphoblastic leukemia, acute leukemia,. chronic lymphocytic leukemia, chronic myelogenous leukemia, osteosarcoma, sarcoma, glioma, astrocytoma, multiple myeloma, glioblastoma multiforme and eppendymoma.

29. The method of claim 28 further comprising administering to the subject a vitamin D analog.

30. The method of claim 29, wherein the vitamin D analog is selected from the group of cholecalciferol, calcifediol, calcitriol, calcipotriol, ergocalciferol, dihydrotachysterol, 1,25-dihydroxyergocalciferol, and 25-hydroxydihydrotachysterol.

31. The method of claim 30, wherein the vitamin D analog is calcitriol.

32. The method of claim 30, wherein the vitamin D analog is calcipotriol.

33. The method of claim 28 further comprising administering to said subject at least one chemotherapy agent.

34. The method of claim 28 further comprising treating said subject with irradiation.

35. The method of claim 28, wherein said cancer is acute lymphoblastic leukemia.

36. The method of claim 28, wherein said cancer is Non Small cell lung cancer.

37. A pharmaceutical composition comprising a pharmaceutically effective dose of a growth factor receptor inhibitor and retinoid wherein:
the retinoid is selected from the group of retinoid D with an alcohol CH₂OH terminal side chain, an ester of retinoid D having an ester bond, an ether of retinoid D having an ether bond, retinoid D where the alcohol CH₂OH terminal side chain is replaced with an aldehyde CHO terminal side chain, retinoid D with a carboxylic acid at the terminal side chain wherein each of the ester bond and the ether bond is formed with the oxygen at the terminal side chain of Retinoid D and wherein retinoid D with the alcohol CH₂OH terminal side chain has the structure: 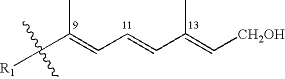
wherein the configuration at the 7-, 9-, 11- and 13-position double bonds is independently Z or E and wherein R₁ is selected from the group of 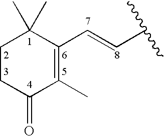
wherein the keto group at the 4position is free or protected, or is replaced by a thioketone group which is free or protected or is replaced by C₁₋₆-alkylidene group; 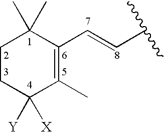
wherein X is selected from the group of hydrogen and C₁₋₆-alkyl and Y is selected from the group of C₁₋₆alkyl, hydroxyl, C₁₋₆-alkoxyl, C₁₋₆-acyloxyl, halide, azide, sulfhydryl, amine and C₁₋₆-alkyl substituted amino and wherein the absolute configuration at the 4-position is independently R or S; 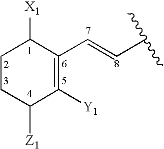
wherein X₁, Y₁ are independently selected from the group of hydrogen, C₁₋₆-alkyl, hydroxyl, C₁₋₆-alkoxyl, C₁₋₆-acyloxyl, halide, azide, sulffiydryl, amine and C₁₋₆-alkyl substituted amino and Z₁ is selected from the group of C₁₋₆-alkyl, hydroxyl, C₁₋₆-alkoxyl, C₁₋₆-acyloxyl, halide, azide, sulfhydryl, amine and C₁₋₆-alkyl substituted amino; 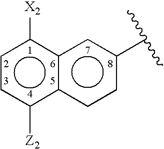
wherein X₂ is selected from the group of hydrogen, C₁₋₆-alkyl-alkyl, hydroxyl, C₁₋₆-alkoxyl, C₁₋₆-acyloxyl, halide, azide, sulfhydryl, amine and C₁₋₆-alkyl substituted amino and Z₂ is selected from the group of C₁₋₆-alkyl, hydroxyl, C₁₋₆-alkoxyl, C₁₋₆-acyloxyl, halide, azide, sulffydryl, amine and C₁₋₆-alkyl substituted amino; 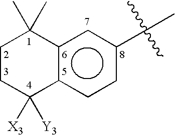
wherein X₃ and Y₃ are independently selected from the group of hydrogens, C₁₋₆-alkyl, hydroxyl, C₁₋₆-alkoxyl, C₁₋₆-acyloxyl, halide, azide, sulfhydryl, amine and C₁₋₆-alkyl substituted amino so long as X₃ and Y₃ are not both hydrogens.

38. The composition of claim 37, wherein the retinoid is selected from the group of 4-oxo-retinol, 4-oxo-retinoic acid, 4-oxo-retinal, and 4-oxo-retinyl ester.

39. The composition of claim 37, wherein the retinoid is all-trans 4-oxo-retinol or an isomer thereof.

40. The composition of claim 37, wherein the retinoid is 4-hydroxy-retinol.

41. The composition of claim 37, wherein the retinoid is 4-methoxy-retinol.

42. The composition of claim 37, wherein said growth factor receptor is EGF receptor.

43. The composition of claim 42, wherein said EGF receptor inhibitor is an EGF receptor antibody.

44. The composition of claim 43, wherein said antibody is C-225.

45. The composition of claim 42, wherein said EGF receptor inhibitor is an inhibitor of tyrosine kinase activity mediated by EGF receptors.

46. The composition of claim 42, wherein said EGF receptor inhibitor is IRESSA®

47. The composition of claim 37, wherein said growth factor receptor is TGF-alpha.
